(19) United States
(12) Patent Application Publication
(10) Pub. No.: US 2025/0241330 A1
(43) Pub. Date: Jul. 31, 2025

(54) PROCESS FOR TREATING PROTEIN-CONTAINING COMPOSITIONS

(71) Applicant: SOCIETE DES PRODUITS NESTLE S.A., Vevey (CH)

(72) Inventors: YUXI DENG, Bern (CH); VERONIKA VOLK, Bern (CH); CHRISTOPH THOMAS WIDMER, Kehrsatz (CH)

(21) Appl. No.: 18/855,014

(22) PCT Filed: Apr. 5, 2023
(86) PCT No.: PCT/EP2023/059024

§ 371 Date: Oct. 8, 2024

(30) Foreign Application Priority Data
Apr. 8, 2022 (EP) 22167472.4

(51) Int. Cl. A23C 9/12; A23C 9/152; A23C 21/02; A23J 3/08; A23J 3/34

(52) U.S. Cl. CPC A23C 9/1216; A23C 9/1522; A23C 9/1528; A23C 21/02; A23J 3/08; A23J 3/343; C12Y 203/02013

(57) ABSTRACT

The invention relates to a process for treating a protein-containing composition. The process includes the provision of a protein-containing composition wherein the proteins comprise milk proteins and said milk proteins comprise at least 90 wt. % β-lactoglobulins. The composition is contacted with a transglutaminase to obtain a cross-linked protein-containing composition. This process enhances not only the heat stability, in particular in presence of calcium but also the texture properties of such protein-containing compositions. The invention also relates to cross-linked protein-containing compositions obtainable or obtained by said process, to a process for preparing dairy products or alternatives thereof with such compositions and to the resulting dairy products or alternatives thereof.

### TECHNICAL FIELD

The present invention relates generally to the field of protein-containing compositions and dairy products or alternatives thereof wherein milk proteins are mostly, preferably consist only of β-lactoglobulins. It also relates to processes for treating such protein-containing compositions and to processes for preparing such dairy products or alternatives thereof.

### BACKGROUND OF THE INVENTION

Milk is an edible liquid produced by the mammary glands of female mammals, such as cows. Milk comprises micro and macronutrients, including a diversity of proteins. Milk proteins can be classified in two main classes: caseins and whey proteins. Caseins represent the majority (80%) of milk proteins and are divided in αS1-, αS2-, β- and κ-casein. Whey proteins, which represent 20% of the milk proteins, typically comprise β-lactoglobulin, alpha-lactalbumin, serum albumin and immunoglobulins.

The major protein in whey protein is β-lactoglobulin. β-lactoglobulin is rich in Leucine. Leucine is one of the 3 essential branched chain amino acids which are used by skeletal muscle to give energy during exercise. Moreover, leucine is also a key amino acid involved in muscle anabolism. For these reasons, β-lactoglobulin stirs up more and more interest from food companies, for example for its use in sport nutrition.

In standard milk ingredient such as milk, milk protein concentrates or whey protein concentrate, β-lactoglobulin is present in a limited amount as it is in combination with other whey proteins and/or caseins. There is an interest for producing food products dairy products or alternatives thereof, including dairy drinks or alternatives thereof, wherein the milk proteins are mostly (i.e. at least 90 wt. %), preferably consist only of β-lactoglobulins.

However, the use of β-lactoglobulins alone or as main milk protein in dairy products or alternatives thereof, in particular dairy drinks or alternatives, entail many disadvantages.

On the one hand, dairy products or alternatives thereof, in particular dairy drinks or alternatives thereof, prepared with β-lactoglobulins as major or sole source of milk proteins exhibit thin texture and poor mouthfeel. The texture of such dairy products or alternatives thereof, in particulardairydrinks or alternatives thereof, is unsatisfactory as theyfail to exhibit similar mouthfeel as dairy products, in particular dairy drinks, which are based on regular milk.

On the other hand, such dairy products or alternatives thereof, in particular dairy drinks or alternatives thereof, exhibit low heat stability. This is explained by low heat-stability of β-lactoglobulins, in particular in presence of calcium. The β-lactoglobulins of these dairy products or alternatives thereof tend to flocculate when heat-treatments at high temperature, such as UHT treatments, are applied, in particular in presence of calcium. For example, this may occur during the manufacturing process. The flocculation is undesirable. Indeed, it impairs the techno-functionality of β-lactoglobulins, for example by decreasing their solubility. Moreover, it negatively impacts the organoleptic profile of resulting dairy products or alternatives thereof, for example by imparting unpleasant inhomogeneous and grainy texture. Furthermore, it may also negatively impact the processability of the dairy products or the alternatives thereof as flocculation may result in the fouling and blocking of the lines.

Hence, there would remain a need to prepare a composition which comprises mostly, preferably only β-lactoglobulins as milk proteins while exhibiting improved thermal stability to high temperatures, in particular in presence of calcium.

It would also be desirable that such a composition imparts good texture, in particular enhanced mouthfeel.

It would also be desirable to prepare dairy products or alternatives thereof, in particular dairy drink or alternatives thereof, which comprise mostly, preferably only β-lactoglobulins as milk proteins while having improved thermal stability to high temperatures, in particular in presence of calcium and good texture, in particular enhanced mouthfeel.

Any reference to prior art documents in this specification is not to be considered an admission that such prior art is widely known or forms part of the common general knowledge in the field.

### SUMMARY OF THE INVENTION

The object of the present invention is to improve the state of the art, and in particular to provide processes, cross-linked protein-containing compositions and dairy products or alternatives thereof that overcomes the problems of the prior art and addresses the needs described above, or at least to provide a useful alternative.

Accordingly, a first aspect of the invention proposes a process for treating a protein-containing composition comprising the steps consisting of:
a) providing a protein-containing composition, wherein the proteins of the protein-containing composition comprise milk proteins, and wherein the milk proteins of the protein-containing composition comprise at least 90 wt. % of β-lactoglobulins, wherein the protein-containing composition is liquid,
b) contacting the protein-containing composition with at least one transglutaminase to obtain a cross-linked protein-containing composition.

A second aspect of the invention proposes a cross-linked protein-containing composition, wherein the proteins of the cross-linked protein-containing composition comprise milk proteins, and wherein said milk proteins of the cross-linked protein-containing composition comprise at least 90 wt. % of β-lactoglobulins and are cross-linked.

A third aspect of the invention proposes a process for preparing a dairy product or an alternative thereof, wherein the proteins of the dairy product or the alternative thereof comprise milk proteins and the milk proteins of the dairy product or the alternative thereof comprise at least 90 wt. % of β-lactoglobulins, said process comprising the steps consisting of:
a) treating a protein-containing composition according to the process of the first aspect of the invention to obtain a cross-linked protein-containing composition or providing a cross-linked protein-containing composition according to the second aspect of the invention,
b) mixing the cross-linked protein-containing composition with calcium or a salt thereof to obtain a food composition,
c) homogenizing the food composition,
d) heat-treating the food composition,
e) optionally, evaporating the food composition,
f) optionally, drying the food composition.

A fourth aspect of the invention proposes a dairy product or an alternative thereof wherein the proteins of the dairy product or the alternative thereof comprise milk proteins, and wherein said milk proteins of the dairy product or the alternative thereof comprise at least 90 wt. % of beta-lactoglobulins and are cross-linked.

It has been observed that cross-linking of β-lactoglobulins allows to improve the heat stability of β-lactoglobulins when exposed to high temperatures, even in presence of calcium. Moreover, the cross-linking of β-lactoglobulins allows to improve texture properties. Indeed, cross-linked β-lactoglobulins impart improved texture, in particular enhanced mouthfeel when used in the preparation of dairy product or alternatives thereof, such as dairy drink or alternatives thereof.

The protein-containing compositions and dairy products or alternatives thereof, which comprise such cross-linked β-lactoglobulins as major, preferably sole source of milk proteins have improved texture, in particular enhanced mouthfeel and have improved heat-stability to high temperatures, even in presence of calcium.

These and other aspects, features and advantages of the invention will become more apparent to those skilled in the art from the detailed description of embodiments of the invention, in connection with the attached drawings.

### BRIEF DESCRIPTION OF THE DRAWINGS

FIG. 1 is a picture showing the stability properties after UHT heat-treatment performed with Rapid Visco Analyzer of the reference milk without calcium of example 5 (A), the reference milk with calcium of example 3 (B) and of the invention milk of example 2 (C). The circle highlights a grain of large size resulting from flocculation.

FIG. 2 shows differential scanning calorimetry measurements of the untreated BLG sample (cf. example 7). Y axis corresponds to enthalpy (mW) and x axis corresponds to temperature (° C.). Curve A corresponds to the first measurement (i.e. first scan) and curve B corresponds to the second measurement (i.e. second scan).

FIG. 3 shows differential scanning calorimetry measurements of the cross-linked BLG sample (cf. example 7). Y axis corresponds to enthalpy (mW) and x axis corresponds to temperature (° C.). Curve A corresponds to the first measurement (i.e. first scan) and curve B corresponds to the second measurement (i.e. second scan).

FIG. 4 shows a picture of the comparative protein-containing composition A with gelled texture obtained after heat treatment at 80° C. for 1 hour.

FIG. 5 is a picture showing the stability properties after UHT heat-treatment performed with Rapid Visco Analyzer of comparative milk A. The circles highlight grains of large size resulting from flocculation.

FIG. 6 shows LDS page to measure the protein molecular weight of the invention milk of example 2. Lane 9 corresponds to LDS page results for invention milk of example 2. Lane 11 corresponds to LDS page results for BLG only sample. Lane 12 corresponds to LDS page results for molecular weight markers. Arrows a to f point out bands for molecular weight markers. a=α-Lactalbumin (14.4 kDa), b=Trypsin (20.1 kDa), c=Carbonic anhydrase (30 kDa), d=Ovalbumin (45 kDa), e=Bovine Serum Albumin (66 kDa), f=Phosphorylase B (97 kDa). Arrow P points out to band for β-lactoglobulin (18 kDa).

### DETAILED DESCRIPTION OF THE INVENTION

As used in the specification, the words “comprise”, “comprising” and the like are to be construed in an inclusive sense, that is to say, in the sense of “including, but not limited to”, as opposed to an exclusive or exhaustive sense.

As used in the specification, the singular forms “a”, “an”, and “the” include plural referents unless the context clearly dictates otherwise.

Unless noted otherwise, all percentages in the specification refer to weight percent, where applicable.

Unless defined otherwise, all technical and scientific terms have and should be given the same meaning as commonly understood by one of ordinary skill in the art to which this invention belongs.

The term “protein” as used herein refers to a polymeric form of amino acids of any length, which can include coded and non-coded amino acids, amino acids that occur in nature and those that do not occur in nature, chemically or biochemically modified or derivatized amino acids, and polypeptides having modified peptide backbones.

The term “recombinant protein” as used herein refers to a protein that is produced in a cell, e.g. recombinant host cell, of a different species or type as compared to the species or type of cell that produces the protein in nature, or that is produced in a cell, e.g. recombinant host cell, at a level at which it is not produced in nature.

The term “non-transglutaminase protein” as used herein refers to all proteins except transglutaminase.

The term “milk proteins” refers to animal milk proteins, which may also be designated as non-recombinant milk proteins and/or recombinant milk proteins, which may be also designated as non-animal milk proteins.

The term “animal milk proteins” or “non-recombinant milk proteins” refers to proteins which come from mammal milk, preferably cow milk (i.e. Bos taurus milk).

The term “recombinant milk proteins” or “non-animal milk proteins” refers to a milk protein that is produced in a cell, e.g. recombinant host cell, of a different species or type as compared to the species or type of cell that produces the milk protein in nature, or that is produced in a cell, e.g. e.g. recombinant host cell, at a level at which it is not produced in nature. In other word, it relates to a protein which can be found inherently in mammal milk, preferably cow milk (i.e. Bos taurus milk), but which is produced recombinantly (e.g., that is produced by a recombinant host cell).

The term “recombinant host cell” as used herein refers to a host cell that comprises a recombinant polynucleotide. Thus, for example, a recombinant host cell may produce a polynucleotide or protein not found in the native (non-recombinant) form of the host cell, or a recombinant host cell may produce a polynucleotide or protein at a level that is different from that in the native (non-recombinant) form of the host cell. It should be understood that such term is intended to refer not only to the particular subject cell but also to the progeny of such a cell. Because certain modifications may occur in succeeding generations due to either mutation or environmental influences, such progeny may not be identical to the parent cell, but are still included within the scope of the term “recombinant host cell” as used herein. Not limitative examples of host cell include bacteria, yeast, fungal cell, plant cell, mammalian cell or any other suitable host cell known in the art.

The term “polynucleotide” as disclosed herein refers to both sense and antisense strands of RNA, cDNA, genomic DNA, and synthetic forms and mixed polymers of the above. A polynucleotide may be modified chemically or biochemically or may contain non-natural or derivatized nucleotide bases. Such modifications include, for example, labels, methylation, substitution of one or more of the naturally occurring nucleotides with an analog, internucleotide modifications such as uncharged linkages (e.g., methyl phosphonates, phosphotriesters, phosphoramidates, carbamates), charged linkages (e.g., phosphorothioates, phosphorodithioates), pendent moieties (e.g., polypeptides), intercalators (e.g., acridine, psoralen), chelators, alkylators, and modified linkages (e.g., alpha anomeric nucleic acids). Examples of modified nucleotides are known in the art (see, for example, Malyshev et al. 2014. Nature 509:385; Li et al. 2014. J. Am. Chem. Soc. 136:826). Also included are synthetic molecules that mimic polynucleotides in their ability to bind to a designated sequence via hydrogen bonding and other chemical interactions. Such molecules are known in the art and include, for example, molecules in which peptide linkages substitute for phosphate linkages in the backbone of the molecule. Other modifications can include, for example, analogs in which the ribose ring contains a bridging moiety or other structure such as the modifications found in “locked” polynucleotides.

The term “recombinant polynucleotide” as used herein refers to a polynucleotide that has been removed from its naturally occurring environment, a polynucleotide that is not associated with all or a portion of a polynucleotide abutting or proximal to the polynucleotide when it is found in nature, a polynucleotide that is operatively linked to a polynucleotide that it is not linked to in nature, a polynucleotide that is altered, or a polynucleotide that does not occur in nature. The term can be used, e.g., to describe cloned DNA isolates, or a polynucleotide comprising a chemically synthesized nucleotide analog. A polynucleotide is also considered “recombinant” if it contains a genetic modification that does not naturally occur. For instance, an endogenous polynucleotide is considered a “recombinant polynucleotide” if it contains an insertion, deletion, or substitution of one or more nucleotides that is introduced artificially (e.g., by human intervention). Such modification can introduce into the polynucleotide a point mutation, substitution mutation, deletion mutation, insertion mutation, missense mutation, frameshift mutation, duplication mutation, amplification mutation, translocation mutation, or inversion mutation. The term includes a polynucleotide in a recombinant host cell's chromosome, as well as a polynucleotide that is not in a recombinant host cell's chromosome (e.g., a polynucleotide that is comprised in an episome). A recombinant polynucleotide in a recombinant host cell or organism may replicate using the in vivo cellular machinery of the recombinant host cell; however, such recombinant polynucleotide, although subsequently replicated intracellularly, is still considered recombinant for purposes of this invention.

The term “identical” as used herein in the context of protein sequences refers to the residues in the two sequences that are the same when aligned for maximum correspondence. The length of sequence identity comparison may be over a stretch of at least 9 amino acid residues, at least 20 amino acid residues, at least 24 amino acid residues, at least 28 amino acid residues, at least 32 amino acid residues, or at least 36 or more amino acid residues. There are a number of different algorithms and methods well known to those skilled in the art that can be used to measure protein sequence identity. By way of example and without being limitative, the percentage identity of two protein sequences may be calculated with CLUSTAL W (version 1.82, version 2), CLUSTAL omega, BLAST, EMBOSS matcher or MULTALIN.

The term “dairy product alternatives” refers to food products wherein the milk proteins comprise, preferably consist only of recombinant milk proteins, in particular recombinant β-lactoglobulins and which generally mimics the texture and visual aspect of standard dairy products. More preferably, such a food product does not comprise any proteins originated from animal, but it may comprise the recombinant form of said proteins originated from animal. Even more preferably, such a food product does not comprise any ingredients originated from animal, except recombinant milk proteins, and recombinant form of other proteins originated from animal. It may comprise non-animal ingredients such as plant ingredients.

The term “standard dairy products” refers to food products which are generally made with milk and/or ingredients derived from milk (e.g. cream) and the milk proteins consist only of animal milk proteins. They generally comprise all type of milk proteins which are inherently found in milk, including caseins & whey proteins.

The term “protein-containing composition” refers to a composition which comprises proteins, including milk proteins. In the frame of the present invention, the milk proteins of the protein-containing composition comprise at least 90 wt. % of β-lactoglobulins, preferably at least 95 wt. %, more preferably at least 98 wt. % of β-lactoglobulins. Even more preferably, milk proteins of the protein-containing composition consist only of β-lactoglobulins.

The term “texturizing agent” refers to ingredients other than milk proteins that contribute to viscosity. Examples include starches (e.g., tapioca starch, corn starch, rice starch, potato starch, cassava starch, corn flour, and the like), pectins, gums (e.g., locust bean gum, carob bean gum, guar gum, and the like), hydrocolloids (e.g., alginate, agar, and the like).

The term “oligomers of β-lactoglobulins” correspond to proteins that comprise, preferably consist of 2 to 10, preferably 2 to 4 β-lactoglobulins. In an embodiment, the β-lactoglobulins in oligomers of β-lactoglobulins are cross-linked inter-molecularly. In particular, the β-lactoglobulins in oligomers of β-lactoglobulins are linked by covalent linkages, in particular by isopeptide bond or amide bond, between the γ-carboxamide group of a glutamine amino acid residue of a β-lactoglobulin, and the free amine group of a lysine amino acid residue of another β-lactoglobulin.

In a first aspect, the invention relates to a process for treating a protein-containing composition.

The process comprises a step a) of providing a protein-containing composition. The protein-containing composition comprises proteins. The proteins of the protein-containing composition comprise milk proteins. The protein-containing composition may have at least 2 wt. % milk proteins, in particular β-lactoglobulins. In a preferred embodiment, the protein-containing composition may have 2 wt. % to 30 wt. %, preferably 2 wt. % to 25 wt. %, more preferably 6 wt. % to 21 wt. %, most preferably 9 to 15 wt. % milk proteins, in particular β-lactoglobulins. With this amount, the composition is a substantial source of milk proteins, in particular β-lactoglobulins. Moreover, this amount ensures enough substrate for an effective enzymatic reaction with transglutaminase.

The protein-containing composition comprises proteins. The proteins of the protein-containing composition comprise milk proteins. The milk proteins of the protein-containing composition comprise at least 90 wt. %, preferably at least 95 wt. %, more preferably at least 98 wt. % of β-lactoglobulins.

In a preferred embodiment, the milk proteins of the protein-containing composition may consist only of β-lactoglobulins. In particular, the protein-containing composition is free from any milk proteins other than β-lactoglobulins. For example, the protein-containing composition is free from caseins.

In an embodiment, the non-transglutaminase proteins of the protein-containing composition may comprise at least 90 wt. %, preferably at least 95 wt. %, more preferably at least 98 wt. % of β-lactoglobulins.

In a more preferred embodiment, the non-transglutaminase proteins of the protein-containing composition may consist only of β-lactoglobulins. In other words, the protein-containing composition does not comprise other proteins than β-lactoglobulins and transglutaminase.

In an embodiment, the β-lactoglobulins are animal β-lactoglobulins and/or recombinant β-lactoglobulins. The recombinant β-lactoglobulins may be at least 80% identical to animal β-lactoglobulins found in a mammal milk, preferably cow milk (i.e. Bos taurus milk).

Preferably, the recombinant β-lactoglobulins may be at least 85%, at least 90%, at least 91%, at least 92%, at least 93%, at least 94%, at least 95%, at least 96%, at least 97%, at least 98%, at least 99%, or at least 99.5% identical to β-lactoglobulins found in a mammal milk, preferably cow milk (i.e. Bos taurus milk).

In an advantageous embodiment, the protein-containing composition is liquid. This enables the enzymatic reaction of step b). Indeed, transglutaminase requires an aqueous liquid system to catalyse the cross-linking reaction.

In an embodiment, the protein-containing composition may be prepared by diluting a concentrated milk protein ingredient into an aqueous liquid, preferably water. The aqueous liquid, preferably water is added in a quantity that enables to reach the targeted milk protein content for the protein-containing composition. The concentrated milk protein ingredient may comprise 60 wt. % to 100 wt. % preferably 75 wt. % to 98 wt. %, more preferably 80 wt. % to 98 wt. % milk proteins, in particular β-lactoglobulins. The milk proteins of the concentrated milk protein ingredient may comprise at least 90 wt. %, preferably at least 95 wt. %, more preferably at least 98 wt. % of β-lactoglobulins. Preferably, the milk proteins of the concentrated milk protein ingredient may consist only of β-lactoglobulins. More preferably, the proteins of the concentrated milk protein ingredient may consist only of β-lactoglobulins. The concentrated milk protein ingredient may be liquid or powder. The concentrated milk protein ingredient may be for example a β-lactoglobulin concentrate or a β-lactoglobulin isolate. The concentrated milk protein ingredient is a concentrated source of β-lactoglobulins. This facilitates the provision of protein-containing composition wherein the milk proteins are mostly, preferably consist only of β-lactoglobulins and having a targeted amount of β-lactoglobulins.

In an embodiment, the protein-containing composition may have a total solids content of at least 2 wt. %. In particular, the protein-containing composition may have a total solids content of 2 wt. % to 30 wt. %, preferably 2 wt. % to 25 wt. %, more preferably 6 wt. % to 21 wt. %, most preferably 9 wt. % to 15 wt. %. The total solids, including proteins, contribute to optimize the enzymatic reaction with transglutaminase by providing sufficient substrate for the enzymatic reaction.

The process further comprises a step b) of contacting the protein-containing composition with at least one transglutaminase to obtain a cross-linked protein-containing composition.

Transglutaminases are a family of enzymes (EC 2.3.2.13) that catalyses the generation of covalent linkages between the γ-carboxamide group of glutamine amino acid residues and the free amine group of lysine amino acid residues present in proteins, such as β-lactoglobulins. When linkages are formed, ammonia is released. The covalent linkages are in particular an isopeptide bond or amide bond. The formation of these covalent linkages between the γ-carboxamide group of glutamine amino acid residues and the free amine group of lysine amino acid residues is called cross-linking. The cross-linking may be intra-molecular, i.e. between glutamine amino acid residues and lysine amino acid residues of the same protein. The cross-linking may be intermolecular, i.e. between the glutamine amino acid residues from a first protein and the lysine amino acid residues from a second protein.

Among the transglutaminases, which can be used in the process of the present invention, one may mention the transglutaminase BDF PROBIND® commercialised by BDF ingredients (Spain).

In an embodiment, the transglutaminase may be allowed to react with the protein-containing composition for 20 minutes to 4 hours at a temperature of 40° C. to 68° C., preferably 48° C. to 65° C. in step b). In a preferred embodiment, the transglutaminase may be allowed to react with the protein-containing composition for 1 to 4 hours at a temperature of 40° C. to 68° C., preferably 48° C. to 65° C. in step b). In a more preferred embodiment, the transglutaminase may be allowed to react with the protein-containing composition for 1 to 2 hours at a temperature of 40° C. to 68° C., preferably 48° C. to 65° C. in step b). These time and temperature conditions contribute to optimize the enzymatic reaction with transglutaminase while avoiding unwanted uncontrolled protein aggregation. Below the temperature range, the enzymatic reaction would be substantially long which is not advantageous for industrial considerations. Above the temperature range, the milk proteins, in particular β-lactoglobulins, and the transglutaminase tend to undergo uncontrolled aggregation leading to undesirable large size protein aggregates. These protein aggregates may negatively impact the functionality of the β-lactoglobulins. They may also impair the enzymatic reaction with the transglutaminase. In particular, this uncontrolled protein aggregation results in protein-containing compositions which are not suitable for use in the preparation of dairy products or alternatives thereof, such as dairy drinks or alternatives thereof. In particular, it may negatively impact the organoleptic properties of the resulting dairy products or alternatives thereof, for example by imparting unpleasant inhomogeneous and grainy structures.

In another embodiment, the transglutaminase is contacted with the protein-containing composition in step b) with a milk protein to transglutaminase weight ratio of 20:1 to 1000:1, preferably 20:1 to 500:1, more preferably 25:1 to 75:1, most preferably of 30:1 to 60:1. This weight ratio ensures a good proportion between the enzyme and its substrate to facilitate an optimal enzymatic reaction with transglutaminase.

It has been discovered that an enzymatic treatment with a transglutaminase improves the heat stability of compositions wherein milk proteins are mostly, preferably consist only of β-lactoglobulins. The transglutaminase treatment cross-links the β-lactoglobulins. This results in β-lactoglobulins having improved heat-stability properties when exposed to high temperature, even in the presence of calcium. They exhibit no or limited flocculation when exposed to high temperature, even in the presence of calcium.

In addition, cross-linked β-lactoglobulins of the invention impart improved texture, in particular enhanced mouthfeel when used in the preparation of dairy product or alternatives thereof, such as dairy drink or alternatives thereof.

In a further embodiment, the process may comprise a step of adding at least one fat component to the protein-containing composition before step b). The fat component according to the invention may be any fat suitable for human consumption. The fat component may be vegetable component, i.e. any fat suitable for human consumption derived from plant material. An example of vegetable fat is vegetable oil. The fat component may be animal component, i.e. any fat suitable for human consumption derived from animal. An example of animal fat is butter. Preferably, the fat component is vegetable fat, in particular vegetable oil. The vegetable oil may be selected from the group consisting of algal oil, almond oil, canola oil, coconut oil, corn oil, cottonseed oil, hazelnut oil, hemp seed oil, grapeseed oil, olive oil, palm oil, peanut oil, rice bran oil, safflower oil, sesame seed oil, soybean oil, sunflower oil, walnut oil, or a combination thereof. The vegetable oil may be in hydrogenated form or in non-hydrogenated form.

In particular, the protein-containing composition and the cross-linked protein-containing composition may comprise at least one fat component as described above. In particular, the protein-containing composition and the cross-linked protein-containing composition may comprise 0.5 wt. % to 7 wt. %, preferably 1 wt. % to 5 wt. %, more preferably 1 wt. % to 3 wt. % fat. In a preferred embodiment, the fat of the protein-containing composition may consist only of vegetable fat. Similarly, the fat of the cross-linked protein-containing composition may consist only of vegetable fat.

The features of the protein-containing composition may apply to the cross-linked protein-containing composition, and vice versa.

In an embodiment, the process may comprise a step of partially or fully inactivating the transglutaminase within the cross-linked protein-containing composition after step b). Preferably, the inactivation step of the process may consist of fully inactivating the transglutaminase within the cross-linked protein-containing composition. This avoids further reaction of the transglutaminase that may negatively impact the sensory properties of the cross-linked protein-containing composition.

The transglutaminase may be inactivated by any state-of-the-art method, for example, by heat treatment, pH lowering or lowering water activity. Preferably, the transglutaminase may be inactivated by heat treatment.

In an embodiment, after the enzymatic treatment step b) or after the inactivation step (if any), the process may further comprise a step of drying the cross-linked protein-containing composition to obtain a cross-linked protein-containing powder. The milk proteins of the cross-linked protein-containing powder may comprise at least 90 wt. %, preferably at least 95 wt. %, more preferably at least 98 wt. % of β-lactoglobulins. In a preferred embodiment, the milk proteins of the cross-linked protein-containing powder may consist only of β-lactoglobulins. In an embodiment, the non-transglutaminase proteins of the cross-linked protein-containing powder may comprise at least 90 wt. %, preferably at least 95 wt. %, more preferably at least 98 wt. % of β-lactoglobulins. In a more preferred embodiment, the non-transglutaminase proteins of the cross-linked protein-containing powder may consist only of β-lactoglobulins. Further details on β-lactoglobulins are provided above. The drying step may be performed by drum drying, roller drying, spray drying or freeze drying. The drying step may also be performed by any other food drying technologies well known in the art. The transformation of the composition into powder may be advantageous as powders have generally good physico-chemical stability and extended shelf-life. The drying step concentrates the proteins, including the β-lactoglobulins. After drying, the resulting cross-linked protein-containing powder may comprise 20 to 80 wt. %, preferably 30 to 50 wt. % milk proteins in particular β-lactoglobulins. In an embodiment, the process may comprise an evaporation step prior to the drying step.

In an embodiment, the cross-linked protein-containing powder may comprise at least one fat component as described above. In an embodiment, the cross-linked protein-containing powder may comprise 1 to 25 wt. %, preferably 4 to 16 wt. % fat. Preferably, the fat of the cross-linked protein-containing powder may consist only of vegetable fat.

In an embodiment, the protein-containing composition is not heat-treated above 75° C., preferably above 80° C. before the step b) of contacting the protein-containing composition with at least one transglutaminase. In particular, the protein-containing composition is not heat-treated for at least 10 seconds, preferably for at least 30 seconds, more preferably for at least 1 minute before said step b).

In another embodiment, the cross-linked protein-containing composition is not heat-treated at a temperature of 70° C. to 95° C., preferably 80° C. to 95° C. just after step b) of contacting the protein-containing composition with at least one transglutaminase. In a preferred embodiment, the cross-linked protein-containing composition is not heat-treated at a temperature of 70° C. to 95° C., preferably 80° C. to 95° C. for 5 seconds to 15 minutes just after step b). For sake of clarity, the cross-linked protein-containing composition may be further heat-treated when used or processed as an ingredient for targeted application. For example, when used/processed in the preparation of food products, in particular dairy products or alternative thereof, the cross-linked protein-containing composition may be heat treated to extend the shelf life of the final food product. However, the present method for treating a protein-containing composition does not comprise a step of heat-treating the cross-linked protein-containing composition at a temperature of 70° C. to 95° C., preferably 80° C. to 95° C. just after step b). The cross-linking of milk proteins, in particular beta-lactoglobulins, with transglutaminase is sufficient to significantly improve the heat stability of protein-containing compositions, in particular when exposed to high temperatures. No heat treatment inducing controlled denaturation of milk proteins is needed after transglutaminase treatment to improve the heat-stability before applying any subsequent heat treatment, including high temperature heat treatment. As a result, the process is simplified but still ensures good heat stability properties to the protein-composition containing.

In an embodiment, the cross-linked protein-containing composition comprises oligomers of β-lactoglobulins having at least 2 β-lactoglobulins, preferably 2 to 10 β-lactoglobulins, more preferably 2 to 4 β-lactoglobulins. In particular, the β-lactoglobulins in the oligomers of β-lactoglobulins are cross-linked, in particular cross-linked inter-molecularly. Further details on the terms “cross-linked” and “cross-linked inter-molecularly” are provided in the second aspect of the invention.

In an embodiment, the cross-linked protein-containing composition comprises proteins, in particular oligomers of β-lactoglobulins, which have a molecular weight which is x times the molecular weight of the β-lactoglobulin used in step a), wherein x is an integer and x is equal or above 2. Preferably, the cross-linked protein-containing composition comprises proteins, in particular oligomers of β-lactoglobulins having a molecular weight which is 2 to 10 times, preferably 2 to 4 times the molecular weight of the β-lactoglobulin used in step a). More preferably, the cross-linked protein-containing composition comprises proteins, in particular oligomers of β-lactoglobulins having a molecular weight which is two times the molecular weight of the β-lactoglobulin used in step a) and/or proteins, in particular oligomers of β-lactoglobulins having a molecular weight which is three times the molecular weight of the β-lactoglobulin used in step a) and/or proteins, in particular oligomers of β-lactoglobulins having a molecular weight which is four times the molecular weight of the β-lactoglobulin used in step a).

In an embodiment, the cross-linked protein-containing composition comprises proteins having a molecular weight of 16 to 180 kDa, preferably 16 to 74 kDa, more preferably 18 to 72 kDa. In another embodiment, the cross-linked protein-containing composition comprises proteins, in particular oligomers of β-lactoglobulins, having a molecular weight of 34 to 180 kDa, preferably 34 to 74 kDa, more preferably 36 to 72 kDa.

In an embodiment, the cross-linked protein-containing composition comprises proteins, in particular oligomers of β-lactoglobulins having a molecular weight 34 to 38 kDa, preferably of 36 kDa and/or proteins, in particular oligomers of β-lactoglobulins having a molecular weight of 52 to 56 kDa, preferably of 54 kDa and/or proteins, in particular oligomers of β-lactoglobulins having a molecular weight of 70 kDa to 74 kDa, preferably of 72 kDa.

In a second aspect, the invention relates to a cross-linked protein-containing composition.

The cross-linked protein-containing composition comprises proteins. The proteins of the cross-linked protein-containing composition comprise milk proteins. The milk proteins of the cross-linked protein-containing composition comprise at least 90 wt. %, preferably at least 95 wt. %, more preferably at least 98 wt. % of β-lactoglobulins.

In a preferred embodiment, the milk proteins of the cross-linked protein-containing composition may consist only of β-lactoglobulins.

In an embodiment, the non-transglutaminase proteins of the cross-linked protein-containing composition may comprise at least 90 wt. %, preferably at least 95 wt. %, more preferably at least 98 wt. % of β-lactoglobulins.

In a more preferred embodiment, the non-transglutaminase proteins of the cross-linked protein-containing composition may consist only of β-lactoglobulins.

The milk proteins, in particular β-lactoglobulins, of the cross-linked protein-containing composition are cross-linked. In particular, part of the glutamine and lysine amino acid residues of the milk proteins, in particular β-lactoglobulins, are cross-linked, i.e. linked by covalent linkages, in particular by isopeptide bond oramide bond, between the γ-carboxamide group of glutamine amino acid residue and the free amine group of lysine amino acid residue.

The milk proteins, in particular β-lactoglobulins, may be at least cross-linked inter-molecularly, i.e. linked by covalent linkages, in particular by isopeptide bond or amide bond, between the γ-carboxamide group of a glutamine amino acid residue of a first milk protein, in particular first β-lactoglobulin, and the free amine group of a lysine amino acid residue of a second milk protein, in particular second β-lactoglobulin. They may also be cross-linked intra-molecularly, i.e. linked by covalent linkages, in particular by isopeptide bond or amide bond, between the γ-carboxamide group of a glutamine amino acid residue and the free amine group of a lysine amino acid residue of the same milk protein, in particular the same β-lactoglobulin.

As mentioned in the first aspect of the invention, β-lactoglobulins which are cross-linked impart improved texture and have enhanced heat stability properties when exposed to high temperatures, even in presence of calcium.

The cross-linked protein-containing compositions of the invention comprising said cross-linked β-lactoglobulins have improved heat stability. In particular, they remain stable and exhibit no or limited flocculation when exposed to high temperature, even in presence of calcium.

In addition, they impart improved texture, in particular enhanced mouthfeel when used in the preparation of dairy product or alternatives thereof, such as dairy drink or alternatives thereof.

The features of the protein-containing composition and the cross-linked protein-containing composition of the first aspect of the invention may apply to the cross-linked protein-containing composition of the present second aspect of the invention, and vice versa.

In a preferred embodiment, the cross-linked protein-containing composition may be obtainable or obtained by the process according to the first aspect of the invention.

The cross-linked protein-containing composition may be a liquid or a powder. When it is a powder, the features of the cross-linked protein-containing powder provided in the first aspect of the invention may apply to the cross-linked protein-containing composition of the second aspect of the invention, and vice versa.

In an embodiment, the cross-linked protein-containing composition comprises oligomers of β-lactoglobulins having at least 2 β-lactoglobulins, preferably 2 to 10 β-lactoglobulins, more preferably 2 to 4 β-lactoglobulins. In particular, the β-lactoglobulins in the oligomers of β-lactoglobulins are cross-linked, in particular cross-linked inter-molecularly. Further details on the terms “cross-linked” and “cross-linked inter-molecularly” are provided above.

In an embodiment, the cross-linked protein-containing composition comprises proteins, in particular oligomers of β-lactoglobulins, which have a molecular weight which is x times the molecular weight of β-lactoglobulin, wherein x is an integer and x is equal or above 2. Preferably, the cross-linked protein-containing composition comprises proteins, in particular oligomers of β-lactoglobulins having a molecular weight which is 2 to 10 times, preferably 2 to 4 times the molecular weight of β-lactoglobulin. More preferably, the cross-linked protein-containing composition comprises proteins, in particular oligomers of β-lactoglobulins having a molecular weight which is two times the molecular weight of β-lactoglobulin and/or proteins, in particular oligomers of β-lactoglobulins having a molecular weight which is three times the molecular weight of β-lactoglobulin and/or proteins, in particular oligomers of β-lactoglobulins having a molecular weight which is four times the molecular weight of β-lactoglobulin.

In an embodiment, the cross-linked protein-containing composition comprises proteins having a molecular weight of 16 to 180 kDa, preferably 16 to 74 kDa, more preferably 18 to 72 kDa. In another embodiment, the cross-linked protein-containing composition comprises proteins, in particular oligomers of β-lactoglobulins, having a molecular weight of 34 to 180 kDa, preferably 34 to 74 kDa, more preferably 36 to 72 kDa.

In an embodiment, the cross-linked protein-containing composition comprises proteins, in particular oligomers of β-lactoglobulins having a molecular weight 34 to 38 kDa, preferably of 36 kDa and/or proteins, in particular oligomers of β-lactoglobulins having a molecular weight of 52 to 56 kDa, preferably of 54 kDa and/or proteins, in particular oligomers of β-lactoglobulins having a molecular weight of 70 kDa to 74 kDa, preferably of 72 kDa.

In a third aspect, the invention relates to a process for preparing a dairy product or an alternative thereof.

The dairy product or the alternative thereof comprise proteins. The proteins of the dairy product or the alternative thereof comprise milk proteins. The milk proteins of the dairy product or the alternative thereof comprise at least 90 wt. %, preferably at least 95 wt. %, more preferably at least 98 wt. % of β-lactoglobulins. In a preferred embodiment, the milk proteins of the dairy product or the alternative thereof may consist only of β-lactoglobulins. In particular, the dairy product or the alternative thereof may be free from any milk proteins other than β-lactoglobulins. For example, the dairy product or the alternative thereof may be free from caseins.

In an embodiment, the milk proteins of the dairy product consist only of animal milk proteins, in particular animal β-lactoglobulins. In an embodiment, the milk proteins of the alternative dairy product comprise, preferably consist only of, recombinant milk proteins, in particular recombinant β-lactoglobulins.

In an embodiment, the non-transglutaminase proteins of the dairy product or the alternative thereof may comprise at least 90 wt. %, preferably at least 95 wt. %, more preferably at least 98 wt. % of β-lactoglobulins.

In a more preferred embodiment, the non-transglutaminase proteins of the dairy product or the alternative thereof may consist only of β-lactoglobulins. In other words, the dairy product or the alternative thereof does not comprise other proteins than β-lactoglobulins and transglutaminase.

The dairy product or the alternative thereof may have at least 0.5 wt. % of milk proteins, in particular β-lactoglobulins. In particular, the dairy product or the alternative thereof may have 0.5 wt. % to 15 wt. %, preferably 1 wt. % to 15 wt. %, more preferably 2 wt. % to 10 wt. %, most preferably 2 wt. % to 5 wt. % milk proteins, in particular β-lactoglobulins.

In an embodiment, the dairy product or the alternative thereof may be selected from the group consisting of cream, creamer, condensed milk, milk powder, milk, a flavoured milk, a smoothie alternative, a milkshake, a powdered milk drink or an alternative thereof.

In a preferred embodiment, the dairy product or the alternative thereof is a dairy drink or an alternative thereof. The dairy drink or the alternative thereof may be selected from the group consisting of creamer, milk, flavoured milk, smoothie, milkshake, powdered milk drink or an alternative thereof.

The process comprises a step a) of treating a protein-containing composition according to the process of the first aspect of the invention to obtain a cross-linked protein-containing composition or a step a) of providing a cross-linked protein-containing composition according to the second aspect of the invention.

In particular, β-lactoglobulins which are cross-linked, namely β-lactoglobulins comprised in the cross-linked protein-containing compositions of the invention, have improved properties. In particular, they impart improved texture and have enhanced heat stability properties when exposed to high temperatures, even in presence of calcium. For these reasons, they are advantageous for the preparation of dairy products or alternatives thereof.

In particular, the dairy products or the alternatives thereof prepared with such cross-linked β-lactoglobulins have an improved texture, in particular an enhanced mouthfeel, compared to dairy products or alternatives thereof prepared with untreated β-lactoglobulins. In addition, said dairy products or alternatives thereof remain stable and exhibit no or limited flocculation, when exposed to high temperature, even in the presence of calcium.

The cross-linked protein-containing composition may be liquid or powder. If the cross-linked protein-containing composition is powder, it may be reconstituted in an aqueous liquid, preferably water, to obtain a liquid cross-linked protein-containing composition before step b).

In an embodiment, the cross-linked protein-containing composition may be diluted in an aqueous liquid, preferably water, to reach a targeted milk protein content, before step b). In particular, after dilution, the cross-linked protein-containing composition may have a milk protein content, in particular β-lactoglobulin content at least 2 wt. %, preferably of 2 wt. % to 30 wt. %, more preferably 2 wt. % to 15 wt. %, most preferably 2 wt. % to 10 wt. %.

The process further comprises a step b) of mixing the cross-linked protein-containing composition with calcium or a salt thereof. Examples of calcium salt include calcium carbonate, calcium chloride, calcium gluconate, calcium lactate, tricalcium phosphate or a combination thereof.

In an embodiment, the food composition and the resulting dairy product or the alternative thereof may comprise 0.01 to 2.5 wt. %, preferably 0.05 to 1.3 wt. %, more preferably 0.1 wt. % to 0.6 wt. % of calcium.

Calcium is a mineral naturally found in milk and which is inherently present in dairy products. Hence, to resemble dairy products, presence of calcium in dairy products or alternatives thereof is paramount. Despite the presence of significant content of calcium, the dairy products or the alternatives thereof of the invention are heat stable and exhibit no or limited flocculation when exposed to high temperatures.

In a preferred embodiment, the cross-linked protein-containing composition may be further mixed with at least one texturizing agent in step b). In particular, the food composition and the resulting dairy product alternative or the alternative thereof may comprise 0.05 to 2 wt. %, preferably 0.1 to 1 wt. %, more preferably 0.1 to 0.5 wt. % of texturizing agent.

In a further embodiment, the cross-linked protein-containing composition may be further mixed with at least one fat component and/or at least one sugar component in step b).

The fat component may be a fat component as described in the first aspect of the invention. The food composition and the dairy product or the alternative thereof may comprise 0.3 to 33 wt. % fat, preferably 0.3 to 15 wt. %, more preferably 1 to 10 wt. %, most preferably 1 to 5 wt. % fat. Preferably, the fat may consist only of vegetable fat.

The sugar components as used herein refers to component that impart or increase the sweet taste in a food product. Examples of sugar component include agave syrup, brown sugar, coconut sugar, maple syrup, rice syrup, oat syrup, corn syrup, dextrose, fructose, glucose, honey, invert sugar, maltose, molasse, sucrose, steviol glycosides and a mixture thereof. The food composition and the dairy product or the alternative thereof may comprise 0 to 15 wt. % sugar component, preferably 0.1 to 15 wt. %, more preferably 1 to 10 wt. %, most preferably 1 to 5 wt. % sugar component.

In an additional embodiment, the cross-linked protein-containing composition may be further mixed with at least one aqueous liquid, preferably water in step b). The food composition and the dairy product or the alternative thereof may comprise 50 wt. % to 95 wt. %, preferably 75 wt. % to 95 wt. % aqueous liquid, preferably water.

In another embodiment, the cross-linked protein-containing composition may be also mixed with one or more additional food ingredients. The additional food ingredients may be any food ingredients suitable for human consumption known to those skilled in the art. In a particular embodiment, the additional food ingredient(s) may be selected from the group consisting of vitamin, minerals other than calcium, fiber, flavouring agent, buffering agent, coloring agent, salt or mixture thereof.

The process also comprises a step c) of homogenizing the food composition. The homogenization step may be applied at a pressure of 50 bar to 700 bar, preferably of 50 bar to 500 bar, more preferably of 50 to 350 bar, most preferably of 100 to 350 bar. The homogenization step may be applied at a temperature from 50° C. to 80° C., preferably from 50° C. to 75° C., more preferably from 55° C. to 70° C.

The process also comprises a step d) of heat-treating the food composition. This step mainly enables to extend the shelf-life of the dairy product or the alternative thereof by limiting the development of undesirable microorganisms, including pathogenic microorganisms. It may also contribute to inactivate the transglutaminase. In that case, an inactivation step is not needed in step a) of said process. The heat-treatment may be applied at a temperature of 70° C. to 150° C. for 3 seconds to 15 minutes, preferably 95° C. to 145° C. for 3 seconds to 5 minutes. For example, the heat-treatment may be an ultra-high temperature (i.e. UHT) treatment, an ultra-pasteurization treatment or a pasteurization treatment. Any food heat treatment technologies known to those skilled in the art may apply.

The dairy product of the invention remains stable along the process, including during the heat-treatment step, even in the presence of calcium. The cross-linked state of the 3-lactoglobulins improves their heat stability, even in presence of calcium.

The heat-treatment step d) may be upstream or downstream to the homogenization step c).

In an embodiment, the process may further comprise after step d), a step of evaporating the food composition. For example, this step may be needed to prepare a condensed milk alternative or an alternative thereof.

In another embodiment, the process may also further comprise after step d) or after the evaporation step (if any), a step of drying the food composition. The drying step may be performed by drum drying, roller drying, spray drying or freeze drying. The drying step may also be performed by any other food drying technologies well known in the art. For example, such a drying step may be needed to prepare a milk powder alternative, powdered milk drink alternative or alternatives thereof.

In an embodiment, the cross-linked protein-containing composition is not heat treated at a temperature of 70° C. to 95° C., preferably 80° C. to 95° C. after step a) and before the step d).

In a preferred embodiment, the cross-linked protein-containing composition is not heat-treated at a temperature of 70° C. to 95° C., preferably 80° C. to 95° C. for 5 seconds to 15 minutes after step a) and before the step d). The cross-linking of milk proteins, in particular beta-lactoglobulins, with transglutaminase is sufficient to significantly improve the heat stability of protein-containing compositions and of the resulting dairy products or alternative thereof, before applying heat treatment of step d) that extends the shelf life of the final dairy products or alternative thereof. No upstream heat treatment inducing controlled denaturation of milk proteins is needed to improve the heat-stability before the heat-treatment of step d).

In an embodiment, the dairy product or the alternative thereof comprise oligomers of β-lactoglobulins having at least 2 β-lactoglobulins, preferably 2 to 10 β-lactoglobulins, more preferably 2 to 4 β-lactoglobulins. In particular, the β-lactoglobulins in the oligomers of β-lactoglobulins are cross-linked, in particular cross-linked inter-molecularly. Further details on the terms “cross-linked” and “cross-linked inter-molecularly” are provided in the second aspect of the invention.

In an embodiment, the dairy product or the alternative thereof comprise proteins, in particular oligomers of β-lactoglobulins, which have a molecular weight which is x times the molecular weight of β-lactoglobulin, wherein x is an integer and x is equal or above 2. Preferably, the dairy product or the alternative thereof comprise proteins, in particular oligomers of β-lactoglobulins having a molecular weight which is 2 to 10 times, preferably 2 to 4 times the molecular weight of β-lactoglobulin. More preferably, the dairy product or the alternative thereof comprise proteins, in particular oligomers of β-lactoglobulins having a molecular weight which is two times the molecular weight of β-lactoglobulin and/or proteins, in particular oligomers of β-lactoglobulins having a molecular weight which is three times the molecular weight of β-lactoglobulin and/or proteins, in particular oligomers of β-lactoglobulins having a molecular weight which is four times the molecular weight of β-lactoglobulin.

In an embodiment, the dairy product or the alternative thereof comprise proteins having a molecular weight of 16 to 180 kDa, preferably 16 to 74 kDa, more preferably 18 to 72 kDa. In another embodiment, the dairy product or the alternative thereof comprise proteins, in particular oligomers of β-lactoglobulins, having a molecular weight of 34 to 180 kDa, preferably 34 to 74 kDa, more preferably 36 to 72 kDa.

In an embodiment, the dairy product or the alternative thereof comprise proteins, in particular oligomers of β-lactoglobulins having a molecular weight 34 to 38 kDa, preferably of 36 kDa and/or proteins, in particular oligomers of β-lactoglobulins having a molecular weight of 52 to 56 kDa, preferably of 54 kDa and/or proteins, in particular oligomers of β-lactoglobulins having a molecular weight of 70 kDa to 74 kDa, preferably of 72 kDa.

In a fourth aspect, the invention relates to a dairy product or an alternative thereof.

The dairy product or the alternative thereof may be a dairy product or an alternative thereof as provided in the third aspect of the invention. In particular, the features of the dairy product or the alternative thereof provided in the third aspect of the invention may also apply to the dairy product or the alternative thereof of the fourth aspect of the invention, and vice versa.

In an embodiment, the dairy product or the alternative thereof may be obtainable or obtained by the process of the third aspect of the invention.

The dairy product or the alternative thereof comprise proteins. The proteins of the dairy product or the alternative thereof comprise milk proteins. The milk proteins of the dairy product or the alternative thereof comprise at least 90 wt. %, preferably at least 95 wt. %, more preferably at least 98 wt. % of β-lactoglobulins.

In a preferred embodiment, the milk proteins of the dairy product or the alternative thereof may consist only of β-lactoglobulins. In particular, the dairy product or the alternative thereof may be free from any milk proteins other than β-lactoglobulins. For example, the dairy product or the alternative thereof may be free from caseins.

In an embodiment, the milk proteins of the dairy product consist only of animal milk proteins, in particular animal β-lactoglobulins. In an embodiment, the milk proteins of the alternative dairy product comprise, preferably consist only of, recombinant milk proteins, in particular recombinant β-lactoglobulins.

In an embodiment, the non-transglutaminase proteins of the dairy product or the alternative thereof may comprise at least 90 wt. %, preferably at least 95 wt. %, more preferably at least 98 wt. % of β-lactoglobulins.

In a more preferred embodiment, the non-transglutaminase proteins of the dairy product or the alternative thereof may consist only of β-lactoglobulins. In other words, the dairy product or the alternative thereof does not comprise other proteins than β-lactoglobulins and transglutaminase.

The milk proteins, in particular β-lactoglobulins, of the dairy product or the alternative thereof are cross-linked. In particular, part of the glutamine and lysine amino acid residues of the milk proteins, in particular β-lactoglobulins are cross-linked. In particular, part of the glutamine and lysine amino acid residues of the milk proteins, in particular β-lactoglobulins, are cross-linked, i.e. linked by covalent linkages, in particular by isopeptide bond or amide bond, between the γ-carboxamide group of glutamine amino acid residue and the free amine group of lysine amino acid residue. The milk proteins, in particular β-lactoglobulins, may be at least cross-linked inter-molecularly, i.e. linked by covalent linkages, in particular by isopeptide bond or amide bond, between the γ-carboxamide group of a glutamine amino acid residue of a first milk protein, in particular first β-lactoglobulin and the free amine group of a lysine amino acid residue of a second milk protein, in particular second β-lactoglobulin. They may also be cross-linked intra-molecularly, i.e. linked by covalent linkages, in particular by isopeptide bond or amide bond, between the γ-carboxamide group of a glutamine amino acid residue and the free amine group of a lysine amino acid residue of the same milk protein, in particular the same β-lactoglobulin.

The dairy product or the alternative thereof may have at least 0.5 wt. % of milk proteins, in particular β-lactoglobulins. In particular, the dairy product or the alternative thereof may have 0.5 wt. % to 15 wt. %, preferably 1 wt. % to 15 wt. %, more preferably 2 wt. % to 10 wt. %, most preferably 2 wt. % to 5 wt. % milk proteins, in particular β-lactoglobulins.

When the dairy product or the alternative thereof is a powder, it may comprise a higher amount of milk proteins, in particular β-lactoglobulins. Especially, it may comprise 5 to 80 wt. %, preferably 20 to 60 wt. % milk proteins, in particular β-lactoglobulins.

As mentioned in the third aspect of the invention, the dairy product or the alternative thereof may comprise 0.01 to 2.5 wt. %, preferably 0.05 to 1.3 wt. %, more preferably 0.1 wt. % to 0.6 wt. % of calcium.

The dairy products or the alternatives thereof of the invention comprising cross-linked β-lactoglobulins as major, preferably sole source of milk proteins have an improved texture, in particular an enhanced mouthfeel compared to dairy products or alternatives thereof comprising untreated β-lactoglobulins as major, preferably sole source of milk proteins. In addition, the dairy products or the alternatives thereof of the invention have improved heat stability. In particular, they exhibit no or limited flocculation at high temperatures, even in presence of calcium.

In an embodiment, the dairy product or the alternative thereof may further comprise calcium or salt thereof, at least one texturizing agent, at least one fat component, at least one sugar component, at least one aqueous liquid and/or at least one additional food ingredient as provided in the third aspect of the invention.

The amount of calcium, texturizing agent, sugar component, fat and aqueous liquid within the dairy product or the alternative thereof are provided in the third aspect of the invention.

In an embodiment, the dairy product or the alternative thereof comprise oligomers of β-lactoglobulins having at least 2 β-lactoglobulins, preferably 2 to 10 β-lactoglobulins, more preferably 2 to 4 β-lactoglobulins. In particular, the β-lactoglobulins in the oligomers of β-lactoglobulins are cross-linked, in particular cross-linked inter-molecularly. Further details on the terms “cross-linked” and “cross-linked inter-molecularly” are provided in the second aspect of the invention.

In an embodiment, the dairy product or the alternative thereof comprise proteins, in particular oligomers of β-lactoglobulins, which have a molecular weight which is x times the molecular weight of β-lactoglobulin, wherein x is an integer and x is equal or above 2. Preferably, the dairy product or the alternative thereof comprise proteins, in particular oligomers of β-lactoglobulins having a molecular weight which is 2 to 10 times, preferably 2 to 4 times the molecular weight of β-lactoglobulin. More preferably, the dairy product or the alternative thereof comprise proteins, in particular oligomers of β-lactoglobulins having a molecular weight which is two times the molecular weight of β-lactoglobulin and/or proteins, in particular oligomers of β-lactoglobulins having a molecular weight which is three times the molecular weight of β-lactoglobulin and/or proteins, in particular oligomers of β-lactoglobulins having a molecular weight which is four times the molecular weight of β-lactoglobulin.

In an embodiment, the dairy product or the alternative thereof comprise proteins having a molecular weight of 16 to 180 kDa, preferably 16 to 74 kDa, more preferably 18 to 72 kDa. In another embodiment, the dairy product or the alternative thereof comprise proteins, in particular oligomers of β-lactoglobulins, having a molecular weight of 34 to 180 kDa, preferably 34 to 74 kDa, more preferably 36 to 72 kDa.

In an embodiment, the dairy product or the alternative thereof comprise proteins, in particular oligomers of β-lactoglobulins having a molecular weight 34 to 38 kDa, preferably of 36 kDa and/or proteins, in particular oligomers of β-lactoglobulins having a molecular weight of 52 to 56 kDa, preferably of 54 kDa and/or proteins, in particular oligomers of β-lactoglobulins having a molecular weight of 70 kDa to 74 kDa, preferably of 72 kDa.

Those skilled in the art will understand that they can freely combine all features of the present invention disclosed herein. In particular, features described for the products of the present invention may be combined with the processes of the present invention and vice versa. Further, features described for different embodiments of the present invention may be combined.

Furthermore, where known equivalents exist to specific features, such equivalents are incorporated as if specifically referred in this specification. Further advantages and features of the present invention are apparent from the figures and non-limiting examples.

### EXAMPLES

### Example 1: Preparation of Cross-Linked Protein-Containing Composition According to the Invention

A cross-linked protein-containing composition was prepared with the treatment of the invention. The cross-linked protein-containing composition comprises only β-lactoglobulin as milk protein source.

A β-lactoglobulin isolate consisting of 92 wt. % β-lactoglobulin, namely Lacprodan® BLG-100 neutral (Arla Foods Ingredients), was diluted with water until reaching a protein content, i.e. β-lactoglobulin content of 10 wt. % to obtain a liquid composition. 1% of a fat component was added to the liquid composition. The liquid composition was then treated with a transglutaminase at 50° C. for 1.5 hours to cross-link the β-lactoglobulins. The resulting cross-linked protein-containing composition was cooled down in an ice bath and then stored before further use.

### Example 2: Preparation of Milk According to the Invention

A milk according to the invention (hereinafter, invention milk) was prepared using the cross-linked protein-containing composition obtained in example 1. The milk comprises only β-lactoglobulin as milk protein source.

First, a milk mixture was prepared by mixing the ingredients of Table 1 below.

TABLE 1

| Ingredients | Quantity (wt. %) |
|---|---|
| Cross-linked protein-containing | 35% |
| composition of example 1 | |
| Calcium carbonate | 0.3% |
| Fat component | 3.5% |
| Sugar | 5% |
| Texturizing agent | 0.5% |
| Water | 55.7% |

After mixing, the milk mixture was heat-treated by UHT treatment at 145° C. for 5 seconds and then homogenized at 150 bars to obtain a milk.

### Example 3: Preparation of Reference Milk

A reference milk was prepared with the same process as provided in example 2. It was also prepared with a similar recipe as the one provided in example 2. However, the 35 wt. % of cross-linked protein-containing composition of example 1 were replaced by 3.5 wt. % of Lacprodan® BLG-100 neutral (Aria Foods Ingredients) which comprises untreated β-lactoglobulin. In addition, the water content was adapted accordingly, i.e. 87.2 wt. % of water was added in the present case.

### Example 4: Preparation of Milk without Texturizing Agent

A texturizing agent-free invention milk was prepared with the same process as provided in example 2.

However, the recipe does not comprise a texturizing agent. In particular, the milk mixture was prepared by mixing the ingredients of Table 2 below.

TABLE 2

| Ingredients | Quantity (wt. %) |
|---|---|
| Cross-linked protein-containing | 35% |
| composition of example 1 | |
| Calcium carbonate | 0.3% |
| Fat component | 3.5% |
| Sugar | 5% |
| Water | 56.2% |

### Example 5: Preparation of Reference Milk without Calcium

A calcium-free reference milk was prepared similarly to the reference milk of example 3.

The only difference is that the recipe of the calcium-free reference milk does not comprise calcium. In particular, the milk mixture was prepared by mixing the ingredients of Table 3 below.

TABLE 3

| Ingredients | Quantity (wt. %) |
|---|---|
| Lacprodan ® BLG-100 neutral | 3.5% |
| Calcium carbonate | 0% |
| Fat component | 3.5% |
| Sugar | 5% |
| Texturizing agent | 0.5% |
| Water | 87.5% |

### Example 6: Assessment of the Thermal Stability of Milks Upon UTH Heat-Treatment in Presence of Calcium

The heat stability of three different milks were assessed:
the reference milk of example 3 which comprises calcium,
the calcium-free reference milk of example 5 which does not comprise calcium, and,
the invention milk of example 2.

To investigate the thermal stability, the milk were prepared with the recipe as mentioned respectively in examples 3, 5 and 2 but the homogenization and the UHTtreatment were not performed as provided in these examples.

In particular, the obtained milk were heat-treated with a UHT heat treatment that was carried out using a Rapid Visco Analyzer (RVA 4800, PerkinElmer, USA). First, 30 g of the different milk was poured in a concentric cylinder CC25-PR pressure cell. Then, the temperature was linearly increased from 50° C. to 140° C. (14° C./min), kept at 140° C. for 1 min and finally decreased linearly to 50° C. The milk were consistently stirred during the heat treatment.

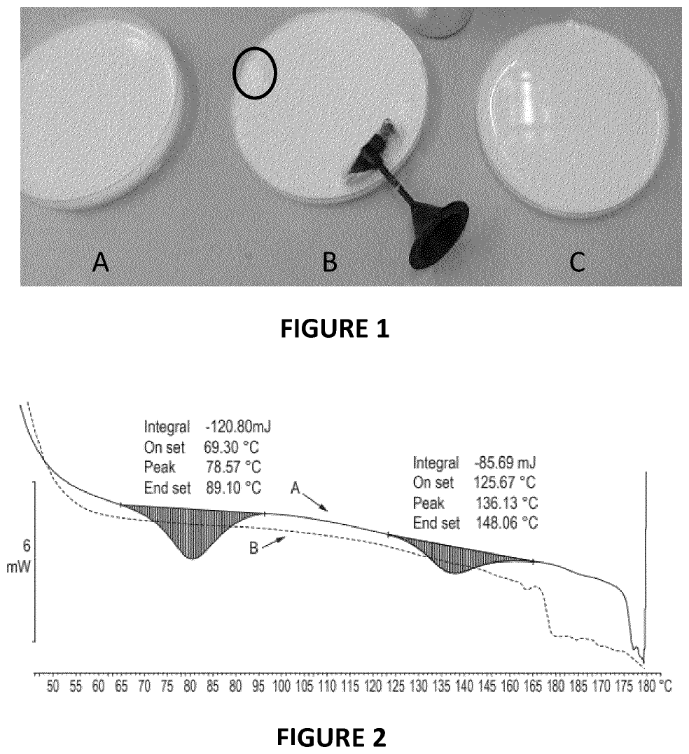

The stability of the milk was assessed by visual inspection after the heat-treatment (FIG. 1). In particular, if the milk are not stable, it can be observed grains visible to the naked eyes. These grains result from flocculation.

The reference milk which comprises calcium underwent flocculation upon UHT treatment and exhibited grains visible to the naked eyes (FIG. 1). On the contrary, the calcium-free reference milk did not undergo flocculation and did not exhibit grains visible to the naked eyes (FIG. 1). Hence, β-lactoglobulins tend to flocculate when calcium is present during the UHT treatment. This flocculation would lead to unpleasant texture in milk, e.g. inhomogeneous texture with perceived grainy texture and poor mouthfeel.

In addition, the invention milk which comprises calcium and wherein the β-lactoglobulins are cross-linked did not undergo flocculation after UHT treatment. No grains can be observed by visual inspection (FIG. 1). Hence, the crosslinking of β-lactoglobulins prior to heat-treatment at high temperatures can enhance the heat stability of β-lactoglobulins in presence of calcium. Without wishing to be bound by theory, it is believed that the crosslinking of glutamine amino acid residues is beneficial to reduce interactions of free calcium ions with the protein molecules. This would result in enhanced heat-stability in presence of calcium.

### Example 7: Differential Scanning Calorimetry Assay

Differential scanning calorimetry was performed on the cross-linked protein-containing composition of example 1, hereinafter cross-linked BLG, and on an untreated protein-containing composition, hereinafter untreated BLG.

The cross-linked BLG sample was prepared as provided in Example 1. The untreated BLG sample was prepared by dissolving Lacprodan® BLG-100 neutral (Arla Foods Ingredients) in water until reaching a protein content, i.e. β-lactoglobulin content of 10%.

The cross-linked BLG and untreated BLG samples were assessed. The differential scanning calorimeter DSC3+(Mettler Toledo, Switzerland) was used for the assessment. Two scans were performed with the following temperature program:
1. 25° C., 10 min,
2. 25° C.-180° C., 10 K/min (first scan),
3. 180° C.-25° C., −10 K/min,
4. 25° C.-180° C., 10 K/min (second scan).

After differential scanning calorimetry, graphs showing enthalpy (y axis) according to the temperature (x axis) were obtained. In these graphs, the onset of the enthalpy peak shows the beginning of protein denaturation, the endset shows the end of denaturation and the peak describes at which temperature the maximum enthalpy was found to denature the protein.

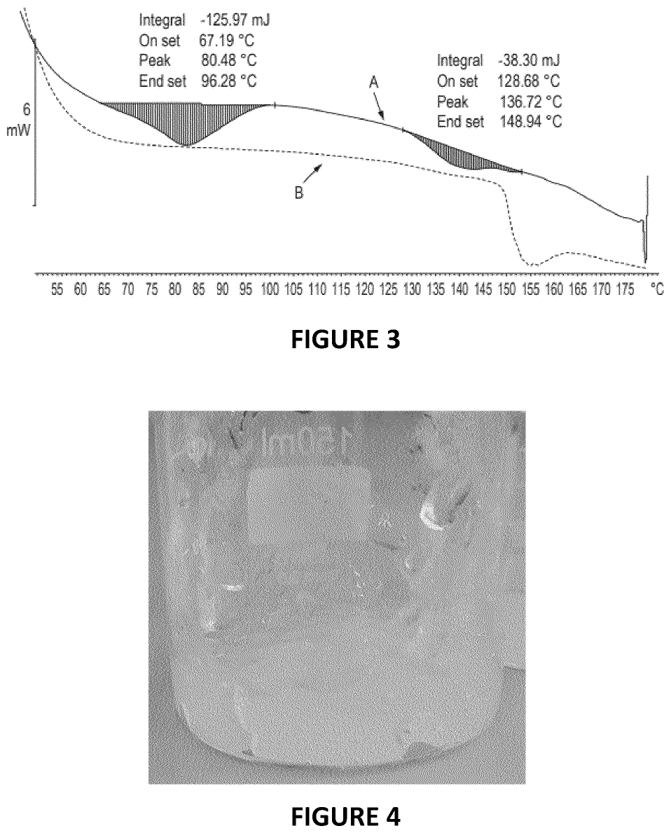

The results are shown in FIGS. 2 and 3. The enthalpy of the crosslinked BLG was modified compared with the untreated BLG. A small shift, in particular an increase of the peak maximum of 1.5° C. was observed for the cross-linked BLG. This may suggest an improvement in the heat stability of β-lactoglobulins for the cross-linked BLG. Low or no enthalpy was observed in the second measurement.

Hence, differential scanning calorimetry data tend to confirm that the cross-linking of β-lactoglobulins improves their heat-stability.

### Example 8: Texture of the milks

The texture, in particular the mouthfeel was assessed for the following milks:
the invention milk of example 2,
the reference milk of example 3, and,
the texturizing agent-free invention milk of example 4.

The mouthfeel of the milk was assessed upon tasting with 12 tasters trained to assess the texture of dairy products, in particular milk.

During tasting, it was noticed that the reference milk had thin texture and poor mouthfeel. In opposition, the invention milk and texturizing agent-free milk had enhanced mouthfeel compared to the reference milk.

It is believed that the crosslinking of β-lactoglobulins by transglutaminase is beneficial for the texture and improves the mouthfeel of milk comprising only β-lactoglobulins as milk proteins.

The invention milk had enhanced mouthfeel compared to texturizing agent-free milk. Hence, it is believed that the mouthfeel was further improved by the addition of texturizing agent to achieve a texture more similar to standard milk.

### Example 9: Impact of Applying Heat Treatment Step Before Transglutaminase Treatment Step (Comparative Milk A)

Prior art document D1=Mariana Battaglin villas-boa et al, “The effect of transglutaminase-induced polymerization in the presence of cysteine on beta-lactoglobulin antigenicity”, international dairy journal, Elsevier applied science, Barking, GB.

Protein treatment involving heat treatment before transglutaminase treatment as disclosed in the prior art document D1 was assessed in the present example.

To do so, a comparative protein-containing composition A was prepared by applying a heat treatment step before attempting to apply an enzymatic treatment with transglutaminase. The comparative protein-containing composition A comprises only 3-lactoglobulin as milk protein source.

A β-lactoglobulin isolate consisting of 92 wt. % β-lactoglobulin, namely Lacprodan® BLG-100 neutral (Arla Foods Ingredients), was diluted with water until reaching a protein content, i.e. β-lactoglobulin content of 10 wt. % to obtain a liquid composition. 1% of a fat component was added to the liquid composition. The liquid composition was then heat treated at 80° C. for 1 hour.

After the heat treatment, the resulting protein-containing composition gelled (FIG. 4). This protein-containing composition in the form of a gel could not be further processed with transglutaminase as transglutaminase cannot be dissolved in the composition as no or limited free water was available.

The resulting protein-containing composition was nevertheless used to prepare a comparative milk A.

The milk mixture of comparative milk A was prepared by mixing the ingredients of Table 4 below.

TABLE 4

| Ingredients | Quantity (wt. %) |
|---|---|
| Comparative protein-containing | 35% |
| composition A | |
| Calcium carbonate | 0.3% |
| Fat component | 3.5% |
| Sugar | 5% |
| Texturizing agent | 0.5% |
| Water | 55.7% |

To investigate thermal stability, the obtained milk mixture of comparative milk A was heat-treated with a UHT heat treatment that was carried out using a Rapid Visco Analyzer (RVA 4800, PerkinElmer, USA). First, 30 g of the milk mixture was poured in a concentric cylinder CC25-PR pressure cell. Then, the temperature was linearly increased from 50° C. to 140° C. (14° C./min), kept at 140° C. for 1 min and finally decreased linearly to 50° C. The milk mixture of comparative milk A was consistently stirred during the heat treatment.

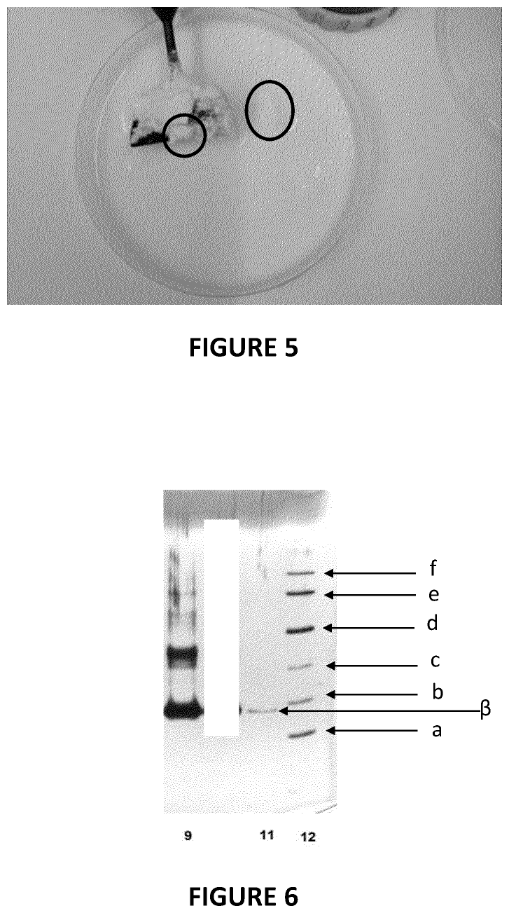

The stability of the comparative milkAwas assessed byvisual inspection afterthe heat-treatment (FIG. 5). In particular, if the milk is not stable, it can be observed grains visible to the naked eyes. These grains result from flocculation.

Comparative milk A exhibits largely aggregated proteins which appear as grains visible to the naked eyes. This suggests that comparative milk A is not stable.

Based on the above, applying a heat treatment before the transglutaminase treatment is not advantageous because:
the heat treatment generates protein-containing composition in the form of gel that cannot be treated or hardly treated with transglutaminase,
the resulting milk is not heat stable and exhibits unsatisfactory grainy texture.

### Example 10: LDS Page Trials

### Materials and Methods

LDS-PAGE (Lithium Dodecyl Sulfate—PolyAcrylamide Gel Electrophoresis) method for the detection of proteins molecular weight (>10 kDa) was performed on samples of invention milk of example 2 and on control samples.

The control samples are the following:
Sample with molecular weight markers which are α-Lactalbumin, β-lactoglobulin, Trypsin, Carbonic anhydrase, Ovalbumin, Bovine Serum Albumin, Phosphorylase B.
Sample comprising only animal β-lactoglobulin which is not cross-linked (BiPro, from Agropur Ingredients, formerly Davisco Food International), hereinafter BLG only sample.

Samples are cleaned by filtration or centrifugation to obtain defatted samples. 65 μL of the defatted samples were denatured with 25 μL lithium dodecyl sulfate (LDS) solution at 95° C., reduced by 10 μL dithiothreitol (DTT).

The prepared samples were then denatured and heated at 95° C. for 5 min to 10 min, cooled down to room temperature, and quickly vortexed.

The prepared samples were then loaded onto a discontinuous NuPAGE 4% to 12% Bis-Tris gel using MES buffer near neutral pH. An electric field was applied on the gel according to electrophoresis principle. Proteins are concentrated in the stacking gel and separated according to their molecular weight in the separating gel. Following separation, gels were fixed, and proteins bands were visualized by silver staining.

Control samples, i.e. sample with molecular weight markers (lane 12) and sample, were prepared similarly as described

### Results

The results are shown in FIG. 6. Lane 9 corresponds to LDS page results for invention milk of example 2. Lane 11 corresponds to LDS page results for BLG only sample. Lane 12 corresponds to LDS page results for molecular weight markers.

It can be observed that the crosslinking increases the molecular weight of the milk proteins. Originally, the beta-lactoglobulin is 18 kDa and after crosslinking we can see the appearance of three new bands above 18 kDa. These molecular weights of these bands correspond to the molecular weight of dimers, trimers and tetramers of beta-lactoglobulins. We can notice that the dimer is the predominant form over the trimer and tetramer. The monomer is still present but part of it has been converted into the dimers, trimers and tetramers after crosslinking.

Although the invention has been described by way of example, it should be appreciated that variations and modifications may be made without departing from the scope of the invention as defined in the claims.

1. Process for treating a protein-containing composition comprising the steps consisting of:
a) providing a protein-containing composition, wherein the proteins of the protein-containing composition comprise milk proteins, and wherein the milk proteins of the protein-containing composition comprise at least 90 wt. % of β-lactoglobulins, wherein the protein-containing composition is liquid, and
b) contacting the protein-containing composition with at least one transglutaminase to obtain a cross-linked protein-containing composition.

2. Process for treating a protein-containing composition according to claim 1, wherein the protein-containing composition comprises at least 2 wt. % milk proteins.

3. Process for treating a protein-containing composition according to claim 1, which comprises a step of adding at least one fat component to the protein-containing composition before step b).

4. Process for treating a protein-containing composition according to claim 1, wherein the transglutaminase is contacted with the protein-containing composition in step b) with a milk protein to transglutaminase weight ratio of 20:1 to 1000:1.

5. Process for treating a protein-containing composition according to claim 1, wherein the transglutaminase is allowed to react with the protein-containing composition for 20 minutes to 4 hours at a temperature of 40° C. to 68° C. in step b).

6. Process for treating a protein-containing composition according to claim 1, wherein the milk proteins of the protein-containing composition consist only of βlactoglobulins.

7. A cross-linked protein-containing composition, wherein the proteins of the cross-linked protein-containing composition comprise milk proteins, and wherein said milk proteins of the cross-linked protein-containing composition comprise at least 90 wt. % of β-lactoglobulins and are cross-linked.

8. A cross-linked protein-containing composition according to claim 7, wherein the milk proteins, of the cross-linked protein-containing composition consist only of β-lactoglobulins.

9. (canceled)

10. A cross-linked protein-containing composition according to claim 7, which comprise proteins having a molecular weight selected from the group consisting of 34 to 38 kDa, 52 to 56 kDa and 70 kDa to 74 kDa.

11. Process for preparing a dairy product or an alternative thereof, wherein the proteins of the dairy product or the alternative thereof comprise milk proteins and the milk proteins of the dairy product or the alternative thereof comprise at least 90 wt. % of β-lactoglobulins, said process comprising the steps consisting of:
a) treating a protein-containing composition according to the steps consisting of:
providing a protein-containing composition, wherein the proteins of the protein-containing composition comprise milk proteins, and wherein the milk proteins of the protein-containing composition comprise at least 90 wt. % of β-lactoglobulins, wherein the protein-containing composition is liquid, and
contacting the protein-containing composition with at least one transglutaminase to obtain a cross-linked protein-containing composition,
b) mixing the cross-linked protein-containing composition with calcium or a salt thereof to obtain a food composition,
c) homogenizing the food composition, and
d) heat-treating the food composition.

12. Process for preparing a dairy product or an alternative thereof according to claim 11, wherein the cross-linked protein-containing composition is further mixed with at least one texturizing agent in step b).

13. Process for preparing a dairy product or an alternative thereof according to claim 11, wherein the cross-linked protein-containing composition is further mixed with at least one fat component and/or at least one sugar component and/or at least one aqueous liquid in step b).

14-16. (canceled)

17. Process for preparing a dairy product or an alternative thereof according to claim 11, wherein the dairy product or the alternative thereof is a dairy drink.

18. Process for preparing a dairy product or an alternative thereof according to claim 11, wherein the milk proteins of the dairy product consist only of animal milk proteins.

19. Process for preparing a dairy product or an alternative thereof according to claim 11, wherein the milk proteins of the alternative dairy product comprise recombinant milk proteins.

20. Process for preparing a dairy product or an alternative thereof according to claim 11, which comprise proteins having a molecular weight selected from the group consisting of 34 to 38 kDa, 52 to 56 kDa and 70 kDa to 74 kDa.
